(19) United States
(12) Patent Application Publication
(10) Pub. No.: US 2005/0266059 A1
(43) Pub. Date: Dec. 1, 2005

(54) Heel moisturizing patch

(76) Inventor: Valerie S. Poss, Redondo Beach, CA (US)

Correspondence Address: Valerie S. Poss, 220 Calle De Madrid, Redondo Beach, CA, 90277, US

(21) Appl. No.: 11/133,985

(22) Filed: May 20, 2005

Related U.S. Application Data
(60) Provisional application No. 60/574,704, filed on May 26, 2004.

(51) Int. Cl. A61K 9/70

(52) U.S. Cl. 424/448

(57) ABSTRACT

The Heel Moisturizing Patch invention solves the problem of dehydrated skin on the heel of the human foot. The Patch is made of a flexible material that is coated with a pressure-sensitive adhesive. The Patch has a non-adhesive area or pad made from foam or other materials in the center. Said pad has been treated with lotion formulations and/or compositions, medications, etc. The Patch stays on the foot by using pressure-sensitive adhesive that sticks to the heel. By leaving the Patch on for a predetermined amount of time, the skin is allowed to rehydrate and remains protected from outside elements. The Patch is particularly adapted at holding creams or other medicaments in direct contact with the skin. Said method is very effective and promotes healing faster than by simply the application of creams and the like to the skin surface in the usual manner in which they are quickly worn away when there is no protective covering for them.

### CROSS-REFERENCE TO RELATED APPLICATIONS

This application claims the benefit of U.S. Provision Patent Application No. 60/574,704 Filed on May 26, 2004.

### FEDERALLY SPONSORED RESEARCH

Not Applicable

### SEQUENCE LISTING OR PROGRAM

Not Applicable

### BACKGROUND OF THE INVENTION

After studying the limitations associated with the application of traditional moisturizing agents, it is apparent that a need exists for a method by which individuals may effectively and conveniently treat dehydrated skin on the heel of the human foot. The methods found in Prior Art require a two step process, by which lotion or other formulations are applied to the skin and then the area is covered with a bandage or cloth. It is the objective of the present invention to provide a better one step method for rehydrating dry skin on the heel of the human foot then what is currently available.

In accordance with this one step method, an adhesive Patch-like bandage with a foam, gauze or other material lining containing lotion formulations and/or treatment compositions therein for maintaining or improving skin health is attached to the skin surface, wherein the skin surface is the heel of an individual. The Patch is left on the heel for a predetermined period of time allowing the moisturizers to absorb directly into the skin. The Patch also prevents further damage from the elements while the patch is in place and promotes faster healing. When the Patch is removed it can easily be disposed of which makes it a cleaner and easier method of treating dry, cracked skin on the heel of the human foot.

The Heel Moisturizing Patch invention solves the problem of dehydrated skin on the heel of the human foot. The present invention provides a method for treating or preventing various dermatological conditions in humans, such as dry, cracked or damaged skin resulting from exposure to sunlight (ultraviolet radiation) and wind, aging effects, general skin dryness.

This Patch-like bandage is made of a predetermined flexible material that is coated with adhesive; like that commonly found on Band-Aids™. The Patch has a non-adhesive area or pad in the middle made from foam, gauze or other predetermined materials. Said area/pad has been treated with lotion formulations and/or treatment compositions, medications, etc. By positioning the patch so that it covers the heel of the foot, the moisturizing agent is allowed to absorb into the skin over time. While the Patch is in place, it is also protecting the heel from further damage from the elements. The Patch stays on the foot by using pressure-sensitive adhesive. The Patch is easily removed and its curved shape helps it fit snuggly to the heel. The Patch may be produced in various sizes and some shape modification may be made as a means for producing an even more effective shape and/or better fit. By leaving the Patch on for a predetermined amount of time, the skin is allowed to rehydrate and remain protected from outside elements. The Patch is particularly adapted at holding creams, gels, ointments, lotions and other medicaments such as moisturizing agents in direct contact with the skin. Said method is very effective and promotes healing faster than by simply the application of creams and the like, to the skin surface in the usual manner in which they are quickly worn away when there is no protective covering for them.

Some existing patents have addressed certain elements of the present invention, but none provide a one step method for the specific purpose of treating damaged skin on the human heel. U.S. Pat. No. #6,506,175-Therapeutic bandage and U.S. Pat. No. #5,109,874—Wound patch and U.S. Pat. No. #3,976,066 Skin Protective device, all have elements claimed herein, but none specifically address the problem of rehydrating dry skin on the heel of human foot. Other patents worth mentioning are listed below:
U.S. Pat. No. #5,750,136 Bio-adhesive composition and patch
U.S. Pat. No. #5,681,580 Patch-type device for iontophoretic teransdermal delivery of insulin
U.S. Pat. No. #5,487,889 Bandage for continuous application of biologicals
U.S. Pat. No. #6,503,525 Absorbent article which maintains or improves skin health
U.S. Pat. No. #6,482,422 Absorbent article which maintains or improves skin health
U.S. Pat. No. #6,316,013 Absorbent article which maintains or improves skin health
U.S. Pat. No. #5,429,591 Absorbent dressing having backing and continuous adhesive layer
U.S. Pat. No. #6,296,862 Absorbent article which maintains or improves skin health
U.S. Pat. No. #6,488,643 Wound healing foot wrap
U.S. Pat. No. #4,139,004 Bandage apparatus for treating burns
U.S. Pat. No. #3,976,066 Skin protective device
U.S. Pat. No. #6,586,652 Absorbent article having a lotioned topsheet
U.S. Pat. No. #5,124,313 Methods of improved skin care and the treatment of dermatological conditions
U.S. Pat. No. #5,682,617 Latex Stocking Bandage
U.S. Pat. No. #5,786,366 Method and Apparatus for Rehydrating Dry skin
U.S. Pat. No. #4,550,035 Cosmetic Applicator useful for skin moisturizing and deodorizing
U.S. Pat. No. #6,653,524 Nonwoven materials with time release additives
U.S. Pat. No. #4,748,976 Anatomical Dressing for Cosmetic Treatments
U.S. Pat. No. #4,961,732 Athlete's foot bandage
U.S. Pat. No. #4,134,399 Skin protective device

### BACKGROUND OF INVENTION-OBJECTS AND ADVANTAGES

Several objects and advantages of the present invention are:
(A) The Patch helps to promote faster healing of dry and damaged skin on the heel of the human foot, because the damaged skin is covered and the moisturizing agent is allowed to absorb into the skin over time
(B) An individual must continually apply the moisturizing agent to his heels throughout the day to achieve the same results as the Patch. Reapplying is time consuming and also messy, as the lotion can come off on socks and clothing
(C) The Patch can be used anytime, anywhere
(D) The method of applying moisturizer without covering it is not as effective as the Patch, because the length of time that moisturizer has to be absorbed into the skin is very limited, which limits the effectiveness of the moisturizing agent
(E) The Patch is a one step method to solve the problem of dry, cracked skin on the heel of the human foot. The Patch is simply “stuck” to the heel and allowed to remain for a period of time. Other methods require a two step process of apply lotion and then covering with a bandage or cloth
(F) Less lotion or other formulations are need with the Patch then other methods because the Patch allows for the first application of lotion or other formulations to be absorbed directly in the skin over time, thus eliminating the need to reapply
(G) While the Patch is in place, it is also protecting the heel from further damage from the elements
(H) This Patch is easily removable
(I) The Patch is secured snuggly to the heel with pressure-sensitive adhesive
(J) The Patch may be produced in various sizes and some shape modification may be made as a means for producing an even more effective design and or better fit.
(K) The Patch is disposable, which is more sanitary and convenient

### SUMMARY OF INVENTION

The Heel Moisturizing Patch invention solves the problem of dehydrated skin on the heel of the human foot. The present invention provides a one step method for treating or preventing various dermatological conditions in humans, such as dry, cracked and/or damaged skin resulting from exposure to sunlight (ultraviolet radiation) and wind, aging effects, general skin dryness. The devices and methods currently available require two steps: applying lotion or formulations and then covering the area with a cloth or bandage.

### BRIEF DESCRIPTION OF THE DRAWINGS

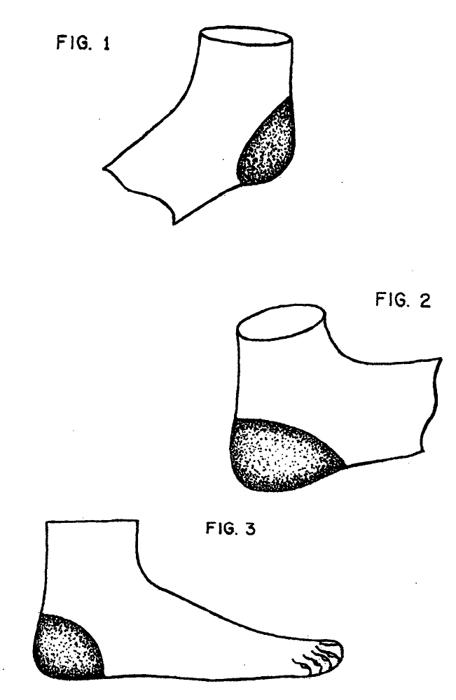

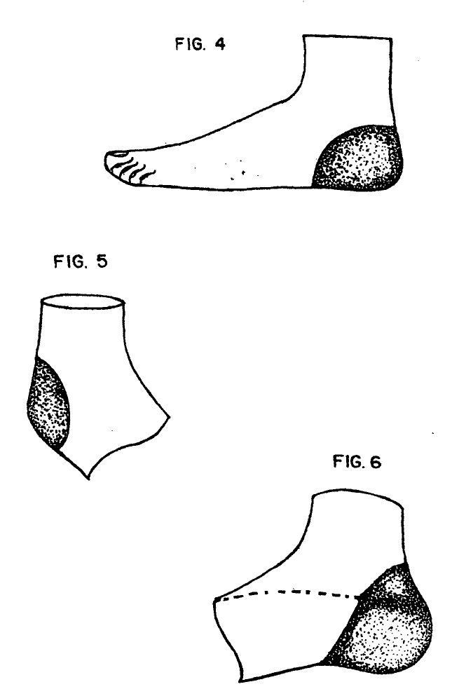

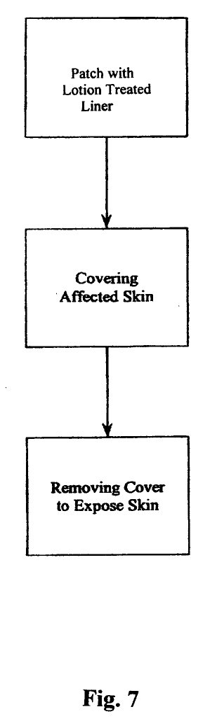

In order to illustrate how the Patch appears as it is directly applied to the heel; reference should be made to FIG. #1-6. FIG. 7 shows is a flow chart outlining the present method. FIG. #8 is a frontal view and illustrates how the patch as it stands alone (not attached to the foot). (A) Shows the natural rubber latex or other flexible materials portion of the Patch that is the outer portion of the patch. (B) Is the pad made from foam, gauze or other materials: that is affixed to (A). The outer peripheral region of (A) is coated with an adhesive material. The adhesive regions that holds the Patch directly over the heel and secure the Patch to the user's skin. (B) Is generally disposed at the center of the adhesive surface. The pad (B) is typically, the non-stick island can be a thin material such as foam, gauze or some other material that has been impregnated with the gel, lotions or other materials, but not limited to; thereon for maintaining or improving skin health. FIG. 9 is a side of the patch when it is not attached to the heel. Design stated, but not limited to the current incarnation.

### DETAILED DESCRIPTION OF INVENTION

The Patch-like bandage is made of a predetermined flexible material such as, but not limited to, a woven or non-woven, synthetic or natural material that is coated with a pressure-sensitive light adhesive on one side. The Patch has a non-adhesive pad made from foam, gauze or other predetermined materials in the center on the same side as the adhesive (see FIG. #8). Said pad has been treated with lotion formulations and/or but not limited to; treatment compositions, medications, etc. Said materials are produced but not limited to, a shape that conforms to the shape of the human heel (see FIG. #1-6 and FIG. #8). The Patch-like bandage is secured temporarily to the heel of the human foot and held there by the pressure-sensitive light adhesive. Applying pressure to the outer ring of adhesive against the skin secures the Patch. To remove the Patch, it is simply pulled always from the skin of the heel until it is completely free of the skin. The Patch can then be disposed of in its entirety. Methods currently available require a two step process, this being:
1) Coating skin with lotion formulations
2) Covering skin with a sock or other forms of cloth

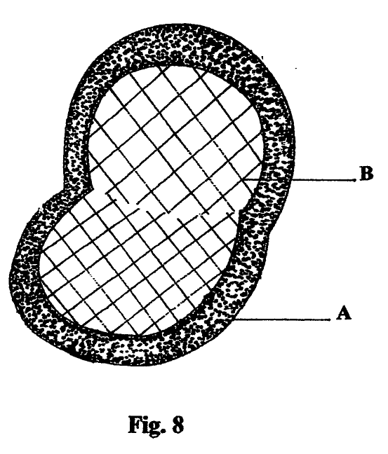

1. A) The Patch-like Bandage is made of a predetermined flexible material such as, but not limited to, a woven or non-woven, synthetic or natural material that is coated with a pressure-sensitive light adhesive. The Patch like bandage has a non-adhesive area or pad made from foam, gauze or other predetermined materials in the center (see FIG. 8). Said pad has been treated with lotion formulations and/or but not limited to; treatment compositions, medications, etc.
B) The present invention provides a one step method for treating or preventing various dermatological conditions on the heels of the human foot
C) The present invention is particularly useful in the application of medicated creams, ointments, lotions and moisturizing agents by holding them in contact with the skin surface of the heel. Because the emollient is substantially immobilized on the surface of the heel, less lotion is required to impart the desired therapeutic or protective lotion coating benefits.
D) The Patch allows for lotion, moisturizing agents, gels, medications, etc to be in direct contact the wearer's skin for a longer absorption period than if lotion is directly applied to the area and left uncovered
E) The limitations of simply applying moisturizing agents to the affected skin become especially problematic when an individual's skin is severely dehydrated and requires immediate, effective treatment. In these instances, the affected skin may remain damaged for many days before enough moisturizing agent is absorb by the affected skin to fully rehydrate the damaged skin. The Patch allows for more moisturizer to be absorbed in a shorted period of time.
F) The Patch protects the area from the elements that caused the damage in the first place, such as dry, cracked or damaged skin resulting from exposure to sunlight (ultraviolet radiation) and wind, aging effects, general skin dryness. The Patch uses adhesives to seal itself to the skin around the heel, forming temporarily barrier over the skin to prevent the further damage from the elements
G) The Patch may be produced in various sizes and shapes as a means for producing an even more effective shape and or better fit over the heel of the human foot
H) The Patch can enhance the health of the skin
I) The Patch can be worn day or night
J) The Patch promotes faster healing
K) The Patch is disposable
L) The Patch can be worn for numerous hours at a time
M) The Patch is a one step method for rehydrating the skin on the heel of the human foot. This makes this method a cleaner/easier method of applying the desired therapeutic or protective lotion coating benefits
N) The pressure-sensitive adhesive allows the Patch to be easily attached to the heel and easily removed
O) The moisturizing agent is allowed to absorb into the skin over time
P) The Patch fits snug and comfortably to the heel
Q) Methods currently available require a two step process, this being: coating skin with lotion formulations and then covering skin with a sock or other forms of cloth. This can be a messy process
R) Existing Patents have addressed certain elements of the present invention, but none provide a One Step method for the specific purpose of treating damaged skin on the human heel.
